(19) United States
(12) Patent Application Publication
(10) Pub. No.: US 2026/0208437 A1
(43) Pub. Date: Jul. 23, 2026

(54) METHOD AND DEVICE FOR PRODUCING AN ARTICLE

(71) Applicant: OTTOBOCK SE & CO KGAA, Duderstadt (DE)

(72) Inventor: Leonard VIER, Duderstadt (DE)

(21) Appl. No.: 19/140,091

(22) PCT Filed: Dec. 18, 2023
(86) PCT No.: PCT/EP2023/086441

§ 371 Date: Jun. 17, 2025

(30) Foreign Application Priority Data
Dec. 20, 2022 (DE) 10 2022 134 212.3

(51) Int. Cl. B29C 64/165; B29K 309/08; B29L 31/00; B33Y 10/00; B33Y 70/10; B33Y 80/00

(52) U.S. Cl. CPC B29C 64/165; B33Y 10/00; B33Y 70/10; B33Y 80/00; B29K 2309/08; B29L 2031/7532

(57) ABSTRACT

The invention relates to a method for producing a three-dimensional articles by way of an additive manufacturing method, wherein at least one manufacturing material is introduced in a flowable state into a support material from at least one introduction opening of an introduction needle and is then hardened, and a stiffening material is introduced into the support material and then hardened.

The invention relates to a method for producing a 3-dimensional object by means of an additive manufacturing process in which at least one manufacturing material is fed in a free-flowing state from at least one feed-in opening of at least one feed-in needle into a supporting material and then cured. The invention also relates to a device for conducting such a method.

Nowadays, many types of additive manufacturing process are known from the prior art and are used to produce a wide range of 3-dimensional objects. Traditionally, additive manufacturing processes are hardly suitable for producing large quantities of the respective objects, as the production of individual objects takes a lot of time. In additive manufacturing processes, especially 3D printing, the object to be produced is built up of a number of very thin layers arranged on top of each other, which are often just a few millimeters thick. The production of large objects in particular is therefore very time-consuming.

In the last few years, a great deal of progress has been made in this area. For example, MIT developed a 3-dimensional printing process that was published in US 2018/281295 A1, for example. Such a method is known as “Rapid Liquid Printing” (RLP). In this case, the object to be produced is made in a container that contains a gel suspension or another material as a supporting material which does not chemically react with the manufacturing material. It serves only to support the manufacturing material as long as it is not yet sufficiently cured. Within the scope of the present invention, “curing” also refers to cross-linking or another process by way of which the free-flowing manufacturing material transforms into a state in which the flow capacity is restricted or no longer present. Such a restriction of flow capacity occurs, for example, due to cross-linking. In the method, the manufacturing material is introduced into the supporting material in a free-flowing state, for example as a liquid or gel, at the desired positions. To this end, at least one feed-in needle is used, which has at least one feed-in opening.

This method has made it possible to also use manufacturing materials that exhibit elastic properties after curing. As a result, it is possible to produce a 3-dimensional object that is elastic by means of an additive manufacturing process.

This is particularly interesting for a wide range of orthopedic objects, such as prosthesis liners. A prosthesis liner is usually made of a silicone or polyurethane. It acts as an intermediate layer between an amputation stump and a prosthesis socket on which further prosthesis parts can be arranged and fixed. The prosthesis liner has an open proximal end, into which the amputation stump is inserted, and a closed distal end. The direction that extends from the distal end to the proximal end or vice-versa is referred to as the longitudinal direction of the prosthesis liner. Prosthesis liners are often standard elements which are not adjusted to the individual circumstances of the respective amputation stump. They often have a circular cross-section that generally tapers from the proximal end to the distal end. This embodiment has the advantage that such a standard liner can be used for many different amputation stumps, meaning it is not necessary to provide a large number of different liners. The cross section may also be oval, ellipsoid or free-form, rather than circular. Each liner, however, has a closed cross-section. In addition to these standard liners, there are also individually produced liners on the market which are adapted to the circumstances of the respective amputation stump. They also have a closed cross-section, which may also be circular, oval, ellipsoid or free-form. Irrespective of the type of liner, the shape of the cross-section may also change depending on the position along the longitudinal direction.

It is known to produce prosthesis liners using an RLP process as this also allows elastic components and objects to be produced. However, the elasticity of a prosthesis liner is preferably not homogeneous. While the liner has to exhibit high elasticity in the circumferential direction so it can be sufficiently widened when applying the liner to the amputation stump, thus achieving a sufficient adhesive effect, the elasticity of the liner along the longitudinal direction of the liner should be significantly lower. Preferably, the liner is either almost or completely inelastic along this direction. The “milking effect” known from the prior art is thus avoided. Such a liner, which exhibits, for example, a different elasticity and stretching properties in the longitudinal direction and the circumferential direction, cannot be manufactured using the RLP processes known from the prior art.

The invention is therefore based on the task of further developing a method in such a way that these disadvantages can be eliminated or at least reduced.

The invention solves the addressed task by way of a method according to the preamble of claim 1, characterized in that a stiffening material is introduced into the supporting material and then cured. The stiffening material exhibits different mechanical properties following curing, in particular a different elasticity, preferably a lower elasticity, than the cured manufacturing material. As a result, the elasticity of the object produced can be changed and influenced in a spatially resolved manner, i.e. to varying degrees at different points.

Advantageously, the stiffening material contains solid particles. The amount and type of these solid particles can be constant throughout all of the stiffening material. In this case, one refers to a homogeneous distribution of solid particles. However, the amount and/or type of solid particles in the stiffening material may vary, in which case it is referred to as an inhomogeneous distribution of solid particles.

The solid particles are arranged in a free-flowing stiffening material when they are introduced into the supporting material. This free-flowing stiffening material can therefore also be referred to as a matrix for the solid particles.

The solid particles preferably comprise fibers. These fibers contain, for example, inorganic fibers, such as boron fibers, silica fibers, carbon fibers, quartz fibers and/or silicate-based fibers, for example basalt fibers, glass fibers or ceramic fibers. Alternatively or additionally, the fibers contain organic fibers, such as aramid fibers and/or carbon fibers, polyester fibers, nylon fibers, polyethylene fibers and/or polymethyl methacrylate fibers. Alternatively or additionally, the fibers contain natural fibers made, for example, of flax, hemp, wood, sisal and/or cotton.

While natural fibers are mainly bonded to the matrix, for example a silicone matrix, by mechanical bonding, inorganic and organic fibers are usually bonded by chemical bonding. In the case of a matrix that either exclusively or predominantly contains a polyurethane, bonding is preferably chemical.

Alternatively or additionally, the solid particles contain glass particles, preferably glass beads. This also allows the mechanical properties of the stiffening material and therefore of the 3-dimensional object to be influenced.

Advantageously, the solid particles, in particular the fibers used, exhibit a lower elasticity than the manufacturing material. Particularly preferably, the fibers also exhibit a lower elasticity than the matrix of the stiffening material. It has been proven advantageous to use inelastic fibers.

The stiffening material is preferably introduced into the support material in such a way that it comes into contact with the previously introduced manufacturing material. Advantageously, at least one feed-in needle with at least one feed-in opening is used to introduce the stiffening material. Preferably, the stiffening material is introduced into the supporting material through the at least one feed-in opening of the at least one feed-in needle in such a way that it comes into contact with the existing manufacturing material immediately upon leaving the feed-in opening. The stiffening material is therefore molded onto the manufacturing material previously introduced into the support material. It is not essential, but advantageous, for all of the stiffening material to be introduced in such a way that it comes into contact with the previously introduced manufacturing material.

In one preferred embodiment, the manufacturing material is not yet fully cured when it comes into contact with the stiffening material. The stiffening material is not cured at this point either, meaning that the two materials, particularly the manufacturing material and the matrix of the stiffening material, can cure together. A chemical bond between the matrix of the stiffening material and the manufacturing material preferably happens in the process, creating an effective and sufficiently stable bond between the two materials. It is advantageous if the matrix of the stiffening material and the manufacturing material are identical. Alternatively, both the matrix of the stiffening material and the manufacturing material can be a silicone and a polyurethane, wherein different silicones and polyurethanes can be used.

Advantageously, at least some of the stiffening material, but preferably all of the stiffening material, is introduced into the supporting material at the same time as the manufacturing material. In particular, if the matrix corresponds to the manufacturing material, the stiffening material can be introduced into the supporting material by the same feed-in needle as the manufacturing material. Preferably, the stiffening material at least partially, but preferably completely, replaces the manufacturing material, at least in some areas. This can be achieved especially easily by adding stiffening elements, such as solid particles, to the manufacturing material. In one particularly preferred embodiment, both the manufacturing material and the stiffening material are introduced into the supporting material using at least one feed-in needle such that preferably at least two feed-in needles are used and situated in the supporting material at the same time.

Preferably, the manufacturing material introduced into the supporting material forms a base body of the 3-dimensional object to be produced, wherein the stiffening material is arranged on said base body. This is preferably done on an outside of the base body, i.e. the side that is not associated with or facing the skin or a body part of the wearer in the finished 3-dimensional object. Thickened areas and additional materials, such as stiffening material, can usually be placed on the outside of the base body without any problems, whereas on the opposite inner side of the base body they often cause problems, pressure points or pain when wearing the finished 3-dimensional object.

Preferably, the fibers include short fibers, long fibers and/or continuous fibers. Short fibers refers to fibers with a maximum length of 1 mm. Long fibers are fibers that are longer than short fibers and have a maximum length of 50 mm. Fibers with a length of more than 50 mm are referred to as continuous fibers. If the stiffening material is introduced into the supporting material by means of at least one feed-in needle, said feed-in needle moves through the supporting material along a predetermined pressure path. In each case, a predetermined amount of stiffening material is introduced into the supporting material along this pressure path. Advantageously, the fibers are aligned at least partially in the direction of movement of the feed-in needle. Preferably, at least 50% of the fibers, preferably over 70% of the fibers, especially preferably over 90% of the fibers in the introduced stiffening material are aligned along the direction of movement of the feed-in needle. The longer the fibers, the more easily they can be aligned by the movement of the feed-in needle. Preferably, particularly long fibers, such as continuous fibers or long fibers, are fed to the stiffening material via a separate feed. As a result, they are already oriented in the stiffening material when it is introduced into the support material. In a storage container, in which the matrix of the stiffening material is mixed with the solid particles, preferably the fibers, and from which the feed-in needle is fed, the solid particles, preferably the fibers, are present in an unoriented manner.

The fibers that are oriented along the direction of movement of the feed-in needle are arranged in such a way that their direction of longitudinal extension forms an angle of at most 30°, preferably at most 20°, especially preferably at most 10°, with the direction of movement. The direction of longitudinal extension of one fiber preferably extends from one end of the respective fiber to the other end of the fiber. As a result, a direction of longitudinal extension can be defined even when the fibers do not extend in a straight line.

The fibers give the stiffening material a high degree of mechanical stability along its direction of longitudinal extension. Conversely, the stability in a direction perpendicular to the direction of longitudinal extension is only slightly influenced or not at all. The selection of the direction of movement of the feed-in needle thus renders it possible to influence the direction along which the stability should be increased, i.e. the structure should be stiffened. In advantageous embodiments of the invention, the direction of movement of the feed-in needle is therefore selected in such a way that different areas or points of the stiffening material are stiffened in different directions. Before producing the object, it is advantageous to determine the expected loads to which the object will be exposed, for example by means of computer simulation. These loads are then used to determine in which areas and in which direction the object should be stiffened against loads. This provides the pressure path which is covered by the feed-in needle during production of the object and which therefore defines the direction of movement of the feed-in needle.

Preferably, the stiffening material creates an auxetic structure. One property of an auxetic structure is that a stretching of the structure in a first direction, for example due to the influence of an external force, results in a stretching in a second direction, for example perpendicular to the first direction.

Preferably, the 3-dimensional object is a liner with a base body that comprises an outer surface, wherein the base body is preferably made of the manufacturing material and wherein the stiffening material is preferably arranged on the outer surface of the base body. Particularly preferably, the stiffening material is arranged on the base body in the distal-proximal direction, preferably in distal-proximal strips. The arrangement of the stiffening material in the distal-proximal direction means that the introduction needle that introduces the stiffening material into the supporting material moves through the supporting material along this direction, i.e. from distal to proximal or vice-versa. Sections of the pressure path which form an acute angle with this direction are also regarded as the distal-proximal direction. As previously explained, the result of using a pressure path with this shape is that fibers in the matrix of the stiffening material form along this direction. If the fibers are inelastic or only slightly elastic fibers, the elasticity of the stiffening material in the distal-proximal direction is reduced by fibers aligned in this way. Since the stiffening material is bonded to the manufacturing material, the mechanical properties, in particular the elasticity, of the manufacturing material bonded to the stiffening material are also reduced in this direction, but are not or only slightly impaired in a direction perpendicular to it, for example in the circumferential direction.

The distal-proximal strips, in which the stiffening material is preferably arranged, can also be referred to as fingers and preferably extend from the distal end of the base body of the liner. Particularly preferably, at least four, preferably at least six, preferably at least eight, preferably at least ten, such fingers or distal-proximal strips are produced. These fingers or distal-proximal strips are preferably arranged equidistantly across the circumference of the base body of the liner.

Preferably, at least one part of the stiffening material, but particularly preferably all of the stiffening material, is arranged on the outside of a part of the object to be produced that is made from the manufacturing material.

The invention also solves the addressed task by way of a device for carrying out a method described here. Such a device has a receptacle, such as a box or a container, which contains the supporting material and in which the 3-dimensional object is produced. The device also has at least one feed-in needle with at least one feed-in opening through which the manufacturing material is introduced into the supporting material. Advantageously, the device also has at least one further feed-in needle with at least one feed-in opening through which the manufacturing material is introduced into the supporting material.

In the following, an embodiment example will be explained in more detail with the aid of the accompanying drawings. They show

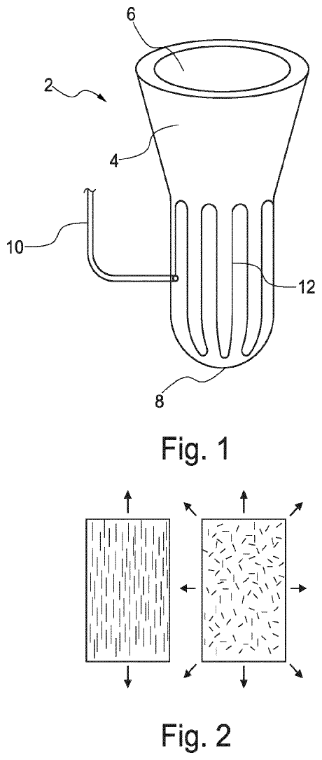

FIG. 1 the schematic representation of a 3-dimensional object produced by means of a method according an embodiment example of the present invention,

FIG. 2 the schematic representation of the effect of fibers on the elasticity and

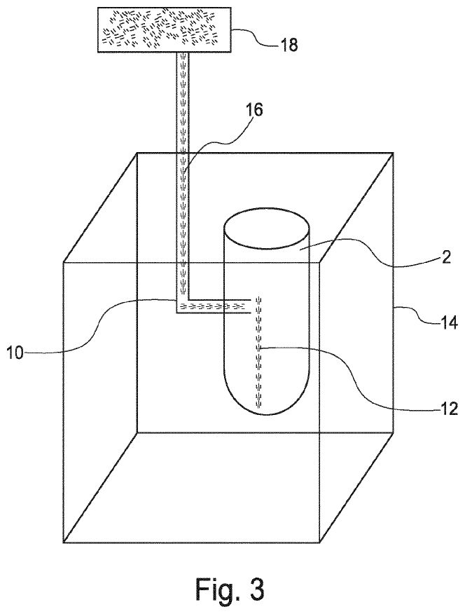

FIG. 3 the schematic representation of a device for performing a method described here.

FIG. 1 depicts a 3-dimensional object comprising a base body 2 that is produced from a manufacturing material in an additive manufacturing process. It has an outer side 4, an open proximal end 6 and a closed distal end 8. The object is preferably a prosthesis liner. A stiffening material is applied to the outer side 4 of the base body 2 via a feed-on needle 10 into the support material, not depicted here. This is done in distal-proximal strips 12.

FIG. 2 demonstrates the influence of fibers on the elasticity of a matrix in which they are arranged. In the right-hand part of FIG. 2 the fibers, depicted by short dashes, are unoriented. The arrows indicate that the elasticity of the matrix is immediately affected, i.e. it is the same in all directions. In the left-hand part of FIG. 2 the fibers are largely, even completely in the embodiment example shown, oriented upwards and downwards along the directions of the arrows. As a result, a stiffening material in which the fibers are oriented in this manner has very restricted elasticity in the direction of orientation, i.e. upwards and downwards, while the elasticity in the perpendicular direction, i.e. from left to right in the representation shown, is barely influenced or not at all.

FIG. 3 schematically depicts a device for performing a method described here. The supporting material is located in a receptacle 14, wherein the former is not depicted in FIG. 3 for the sake of clarity. The feed-in needle 10 is shown inside the receptacle 14, the former being used to introduce stiffening material 16 from a reservoir 18 into the supporting material and the receptacle 14. The fibers of the stiffening material 16 that are depicted as short dashes are unoriented in the reservoir 18 and are oriented along the direction of movement of the needle on their way through the feed-in needle 10. The schematic representation in FIG. 3 seems to indicate that the feed-in needle 10 cannot be moved relative to the receptacle 14. This, however, is not correct and merely due to the clarity of the representation. The feed-in needle 10 can preferably be moved along three independent directions, which are preferably perpendicular to each other, wherein combinations of these directions are also possible. Inside the receptacle 14 there is a base body 2, which has been produced from a manufacturing material in a previous step in the method. A distal-proximal strip 12 can be seen on the outer side of the base body which is made of the stiffening material 16, illustrated by fibers depicted as short dashes.

### REFERENCE LIST

2 base body
4 outer side
6 proximal end
8 distal end
10 feed-in needle
12 distal-proximal strip
14 receptacle
16 stiffening material
18 reservoir

1. A method for producing a 3-dimensional object by an additive manufacturing process, comprising:
feeding at least one manufacturing material in a free-flowing state from at least one feed-in opening of at least one feed-in needle into a supporting material;
curing the at least one manufacturing material fed into the supporting material;
introducing a stiffening material into the supporting material; and
curing the stiffening material introduced into the supporting material.

2. The method according to claim 1, wherein the stiffening material contains solid particles.

3. The method according to claim 2, wherein the solid particles contain fibers.

4. The method according to claim 2 wherein, the solid particles comprise glass particles.

5. The method according to claim 1 wherein introducing the stiffening material into the supporting material is performed such that the stiffening material comes into contact with the at least one manufacturing material after the at least one manufacturing material has been fed into the supporting material.

6. The method according to claim 1 wherein the at least one manufacturing material is not yet fully cured when it comes into contact with the stiffening material.

7. The method according to claim 1 wherein at least some of the stiffening material is introduced into the supporting material at the same time as the at least one manufacturing material is fed into the supporting material.

8. The method according to claim 1 wherein the at least one manufacturing material fed into the supporting material forms a base body on which the stiffening material is arranged.

9. The method according to claim 1 wherein the stiffening material is introduced into the supporting material by a second feed-in needle.

10. The method according to claim 3 wherein the fibers contain short fibers, long fibers, and/or continuous fibers.

11. The method according to claim 1 further comprising configuring the 3-dimensional object as a liner with a base body that comprises an outer surface.

12. The method according to claim 11, wherein the stiffening material is arranged on the base body in a distal-proximal direction.

13. The method according to claim 1 wherein the steps are performed such that least some of the stiffening material is arranged on an outer side of a part of the 3-dimensional object produced.

14. A device for carrying out a method according to claim 1.

15. The method of claim 3 wherein the fibers are selected from the group consisting of inorganic fibers, ceramic fibers, quartz fibers, organic fibers, carbon fibers, and natural fibers.

16. The method of claim 2 wherein the solid particles include one or more of glass fibers and glass beads.

17. The method according to claim 7 wherein all of the stiffening material is introduced into the supporting material at the same time the at least one manufacturing material is fed into the supporting material

18. The method of claim 11 wherein the base body is made of the at least one manufacturing material and wherein the stiffening material is arranged on the outer surface.

19. The method of claim 12 wherein the stiffening material is arranged as distal-proximal strips on the base body.
